(19) United States
(12) Patent Application Publication
(10) Pub. No.: US 2010/0236548 A1
(43) Pub. Date: Sep. 23, 2010

(54) Soft bite block and method for making soft bite block

(76) Inventors: Nancy Reis, (US); Susan Condry, (US); Diana Roloff, (US)

Correspondence Address: Nancy Reis, c/o Pearce & Durick, 314 East Thayer Avenue, Suite 300, Bismarck, ND, 58501, US

(21) Appl. No.: 12/383,268

(22) Filed: Mar. 23, 2009

(51) Int. Cl. A61M 16/00; A62B 9/06; B32B 38/00

(52) U.S. Cl. 128/200.26; 128/207.14; 156/242

(57) ABSTRACT

A soft bite block device for preventing contact between upper and lower teeth of an anesthetized patient. The device includes an elongate roll of cotton gauze encircled by permeable tape so as to maintain the rolled gauze as a monolithic structure. The device is disposable, absorbent and aseptic or sterile. The device is contained within an aseptic or sterile package for the protection of the patient.

### BACKGROUND OF THE INVENTION

1. Field of the Invention

The present invention relates generally to a patient airway bite block and, more particularly, to a soft bite block used together with laryngeal mask airways (LMAs), oral endotracheal tubes (ETT), and similar patient airways. The soft bite block is contained within a sterile or aseptic package.

2. Description of Related Art

Patients undergoing general anesthesia must have their airways secured in order to assure adequate ventilation. This is often accomplished through the use of an LMA or an ETT. The LMA functions in place of either a patient face mask or an endotracheal tube. LMAs are comprised of a distal portion which is a cuffed disc-like device which fits around the larynx in the posterior hypopharynx and a more proximal portion which is analogous to an endotracheal tube. LMAs are placed in anesthetized patients blindly and the exiting tube portion is positioned directly in the mid-line of the mouth.

Use of an LMA or an ETT in anesthetized patients pose several serious problems. First, patients may bite down on the tube portion of the device and cause airway obstruction. This can lead very quickly to hypoxemia (i.e., dangerously low levels of oxygen in the blood) of the patient. Second, such biting by the patient's incisors can cause actual severing of the LMA or ETT and subsequent loss of control of the airway. Third, secretions tend to accumulate in the back of the throat during general anesthesia because there is a loss of the normal swallowing reflexes in anesthetized patients. In a lightly anesthetized patient, or in a patent that is awakening from general anesthesia, such secretions can cause laryngospasm and subsequent airway closure. Therefore, it is best to keep the teeth apart with the soft bite block to allow suctioning of secretions. Because of the above-described problems encountered using an LMA or an ETT, it is recommended to place a bite block between the teeth of the anesthetized patient.

Conventional oral airways, which are usually used in patients anesthetized with their airway secured with oral endotracheal tubes, are not suitable for use with LMAs because such devices seat themselves directly in the mid-line of the mouth and thus compete for the space where the tube portion of the LMA exits the mouth. In addition, the posterior portion of the oral airway which is used to hold the tongue forward when used with an endotracheal tube impinges on the cuffed portion of the LMA in the hypopharynx resulting in the cuff not functioning properly.

Among the solutions practitioners have employed to provide bite blocks for patients with LMAs include the modification of other products which are intended for completely different uses. For example, a bite guard for use with gastroscopy patients has been described for use as a bite block. This device is not suitable because (1) it is not designed for use with LMAs and is not sized appropriately, (2) it seats in the center of the mouth, (3) it is not safe for patients with frontal dental bridge work since this is the area that will bite down on the device, and (4) it has no handle and therefore can be lost in the back of the patient's throat.

U.S. Pat. No. 4,425,911 (Luomanen et al.) discloses a bite-block for intubated endotracheal tubes. The bite block includes a body having a substantially rectangular cross-section, a face plate joined to the body at the exterior end thereof, and a projection extending laterally from the body on one side thereof. The body is provided with a longitudinally extending centrally located U-shaped channel open at the top, and a pair of continuously extending open-sided U-shaped channels on either side of the center channel. Upper and lower surfaces of the projection extend substantially perpendicular to the side of the main body, with the projection terminating in a flange. Ridges or steps are provided on the upper and lower surfaces and are configured for complementary engagement with the patient's canines, bicuspids and molars.

However, there are a number of drawbacks in the bite block of Luomanen et al. In particular, the upper and lower teeth contacting surfaces are not angled to provide for the jaw to be opened as wide as possible. Moreover, the face plate of the Luomanen et al. device provides the potential for injury or damage to the incisors or lips by, for example, pressure exerted on the face plate causing the device to be pushed posteriorly and in turn easily damaging the incisors. The face plate also prevents further posterior movement of the device. Further still, the Luomanen et al. device is designed to hold an endotracheal tube precisely and tightly in place, whereas the LMA breathing tube portion requires a slight freedom of movement in order to perform adjustments in the cuff volume thereof. The Luomanen et al. device includes an integral portion designed to keep the tongue from slipping back into the patient's throat. Accordingly, as a patient awakens from a general anesthetic, the device must be removed prior to the return of the pharyngeal reflexes (i.e., gagging, regurgitation, etc.). The Luomanen et al. device lacks any type of handle for positioning and removing the device within and from, respectively, the patient's mouth.

U.S. Pat. No. 2,708,931 (Freedland) and U.S. Pat. No. 2,694,397 (Herms) disclose a mouth guard and a mouth prop, respectively, for patients undergoing shock therapy, epileptics, or other convulsive condition. However, such devices are unsuitable for use with LMAs because they are bilateral in configuration and therefore partly occupy the center of the mouth which would preclude the use of an airway product such as an LMA in conjunction therewith. Moreover, both of the devices are reusable and include handles which are positioned directly in a mid-line location that would interfere with a device such as an LMA. Also, with respect to the portion actually engaged by the patient's teeth, there is no angulation to provide for progressive opening of the jaw.

U.S. Pat. No. 5,009,595 (Osborn) discloses a mouth prop for dental patients. The device includes lateral flanges which keep the device from moving medially in the mouth. The flanges are molded specifically at a right angle from the bite block itself and thus are not angled away from the gum area. Furthermore, there are no flanges on the lingual (tongue) side so that a dentist can work on the medial aspects of the teeth in the area of the mouth prop. A hole is formed so that the practitioner can place dental floss or a cord through the device as a safety feature so as not to loose the device into the back of the patient's throat.

U.S. Pat. No. 5,174,284 (Jackson) discloses a bite block which is used specifically in awake or sedated patients undergoing endoscopic procedures such as gastroscopy. The bite block covers virtually the entire mouth and thus would be totally unsuitable for use with an LMA or an ETT.

The prior art bite blocks have failed to provide a bite block which is suitable for use with an LMA or an ETT, is soft, absorbent, inexpensive to manufacture and is contained within an aseptic or sterile package so as to avoid the transfer of harmful bacteria or microorganisms into the patient.

### SUMMARY OF THE INVENTION

The present invention provides a bite block which overcomes the above-discussed drawbacks of the conventional devices. More specifically, the present invention provides a disposable, absorbent and soft bite block, preferably for use with laryngeal mask airways (LMAs) or oral endotracheal tubes (ETT). Furthermore, the soft bite block is contained within an aseptic or sterile package for the protection of the patient.

In accordance with the present invention, a bite block is provided for use by a human patient and comprises an absorbent inner core and an outer cover. The inner core is comprised of a substantially cylindrical mass of compressed absorbent fibers. The absorbent fibers may be selected from a variety of natural liquid-absorbing materials commonly known in the art and used in absorbent articles such as cellulosic materials for example cotton, rayon (viscose), wood pulp, crepped cellulose wadding, cross-linked cellulosic fibers. The absorbent inner core materials can be formed into a fabric, web, or batt that is suitable for use in the absorbent material by any suitable process such as airlaying, carding, wetlaying, hydroentangling, needling, or other known techniques.

The outer cover is preferably made from a smooth material to avoid friction against the walls of the oral cavity during insertion of the bite block, retention of the bite block and removal of the bite block. The outer cover may be made from at least one layer of binding material such as tape, mesh, or certain thermoplastics for example polymers, for example polyesters, such as polycarbonates, for example nylon, or such as poly(methyl metaacrylate).

The bite block of the present invention may be any suitable size and thickness which is suitable for insertion in the patient's mouth and adequate to hold the teeth spaced apart sufficient under biting load to allow the use of a tube inserted between the teeth. The length of the bite block of the present invention should be such as to allow a portion to be placed between the teeth in the oral cavity from the molars to the incisors having sufficient length left over to be extra-oral to provide for grasping for insertion and removal and to allow monitoring of the location of the bite block during or after surgery.

Means is provided for easily withdrawing the bite block from the patient's oral cavity. The withdrawal means will be joined to the bite block and is graspable for removal after use. The withdrawal means may be joined to any suitable location on the bite block. In the typical manufacturing process, the withdrawal means would be attached to the absorbent inner core before the absorbent inner core is subject to compression. The withdrawal means can take on the form of a ribbon, tab, loop or can consist of an extra-oral section of the soft bite block.

Therefore, a general object of the present invention is to provide a soft bite block which can be easily placed between a patient's teeth in the oral cavity.

A further objective of the present invention is to provide a soft bite block which can be used with a laryngeal mask airways (LMAs) or oral endotracheal tubes.

It is the further object of the present invention to provide a soft bite block which is comprised of an absorbent inner core.

It is the further object of the present invention to provide a soft bite block which will result in less tooth damage when compared to the use of a rigid bite block.

It is the further object of the present invention to provide a soft bite block which is contained within an aseptic or sterile package so as to avoid the transfer of harmful bacteria or microorganisms into the patient.

These, and other, aspects and objects of the present invention will be better appreciated and understood when considered in conjunction with the following description and the accompanying drawings. It should be understood, however, that the following description, while indicating preferred embodiments of the present invention, is given by way of illustration and not of limitation. Many changes and modifications may be made within the scope of the present invention without departing from the spirit thereof, and the invention includes all such modifications.

Such modifications can, among others, include the soft and absorbent materials used and the means and methods for packaging the soft bite block.

### BRIEF DESCRIPTION OF THE DRAWINGS

FIG. 1 is a perspective view of the soft bite block in accordance with the preferred embodiments of the present invention.

FIG. 2 is a perspective view of the inner core blank being formed into the inner core in accordance with the preferred embodiments of the present invention.

FIG. 3 is a perspective view of the inner core when formed from an inner core blank in accordance with the preferred embodiments of the present invention.

FIG. 4 is a perspective view of cotton gauze being rolled to form the inner core in accordance with the preferred embodiments of the present invention.

FIG. 5 is a perspective view of the inner core when formed from cotton gauze in accordance with the preferred embodiments of the present invention.

FIG. 6 is a top perspective view of the soft bite block positioned within a patient's mouth.

FIG. 7 is a perspective view of the outer cover in accordance with the preferred embodiments of the present invention.

FIG. 8 is a perspective view of the soft bite block containing the withdrawal means and the soft bite block positioned within a patient's mouth.

FIG. 9 is a perspective view of the soft bite block contained within a aseptic package and a forward view of the package in accordance with the preferred embodiments of the present invention.

FIG. 10 is a partial perspective view of the package containing unsealed opposing flaps.

### DETAILED DESCRIPTION OF THE PREFERRED EMBODIMENTS

The present invention and the various features and advantageous details thereof are explained more fully with reference to the non-limiting embodiments described in the following description.

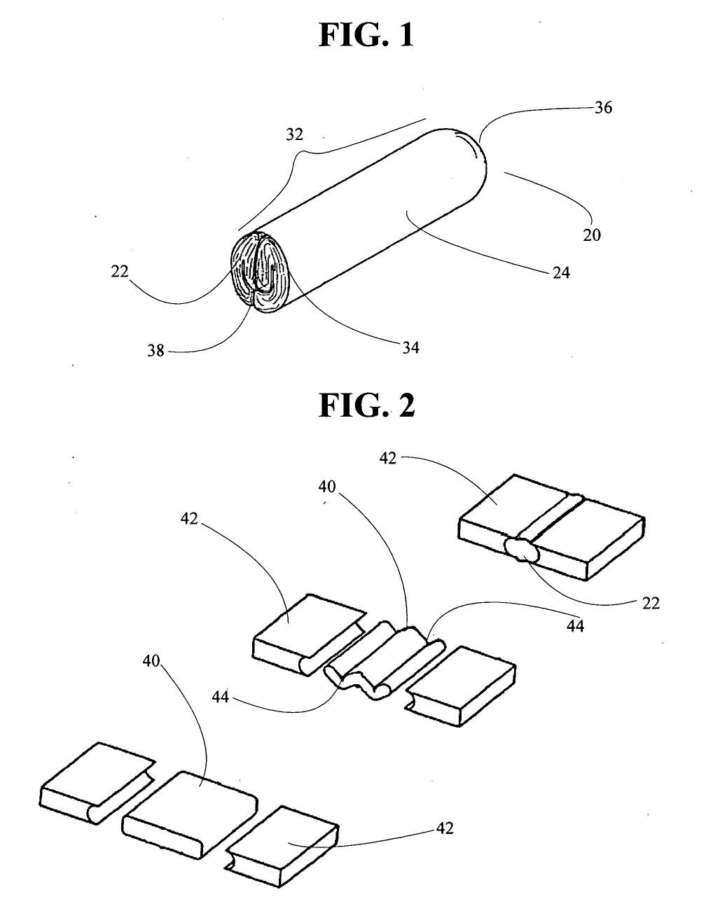

FIGS. 1 through 10 show a device of the present invention which provides a disposable, absorbent and soft bite block 20, preferably for use with laryngeal mask airways (LMAs) or oral endotracheal tubes. Furthermore, the soft bite block 20 is contained within an aseptic or sterile package 28 for the protection of the patient 30.

I. Bite Block of the Present Invention

Referring to FIG. 1, the soft bite block 20 has an axial length 32, a circumference 34 and a first end 36 with an opposed second end 38. The bite block 20 may be any suitable size and thickness which is suitable for insertion in the patient's 30 mouth. The soft bite block 20 is comprised of an inner core 22 comprising a compressed absorbent material, an outer cover 24 comprising at least one layer of material, and a withdrawal 26 means for easily removing the soft bite block 20 from the patient's 30 mouth.

A. Absorbent Inner Core

The absorbent inner core 22 may be selected from a variety of natural liquid-absorbing materials commonly known in the art and used in absorbent articles such as cellulosic materials for example cotton, rayon (viscose), wood pulp, crepped cellulose wadding, cross-linked cellulosic fibers. Also synthetic materials such as for example polyester fibers, polyolefin fibers, absorbent sponges, absorbent foams, absorbing gelling-materials can be used as absorbent materials according to the present invention. In a preferred embodiment the absorbent material comprises fibrous material. Furthermore, the absorbent fibrous material comprises natural and/or synthetic fibers.

In a particularly preferred embodiment the absorbent inner core 22 comprises rayon (viscose) fibers and/or cotton fibers. Starch-based polymers are also suitable as inner core material. Polymers for example including as a first component a hydroxyl-functional polyester and as the second component a natural polymer such as starch, guar gum, cellulose or the like are particularly useful as inner core material according to the present invention. In a preferred embodiment the polymer is biodegradable. The above list of materials that are suitable as inner core 22 material is non-limiting. The listed materials may be combined to form the absorbent inner core 22. The absorbent inner core 22 materials can be formed into a fabric, web, or batt that is suitable for use in the absorbent material by any suitable process such as airlaying, carding, wetlaying, hydroentangling, needling, or other known techniques.

FIG. 2 exemplifies a inner core 22 blank 40 comprised of a batt of absorbent material in an uncompressed state. The blank 40 may be any suitable shape, size, material or construction. In the preferred embodiment of the present invention, the blank 40 is generally square or rectangular shaped.

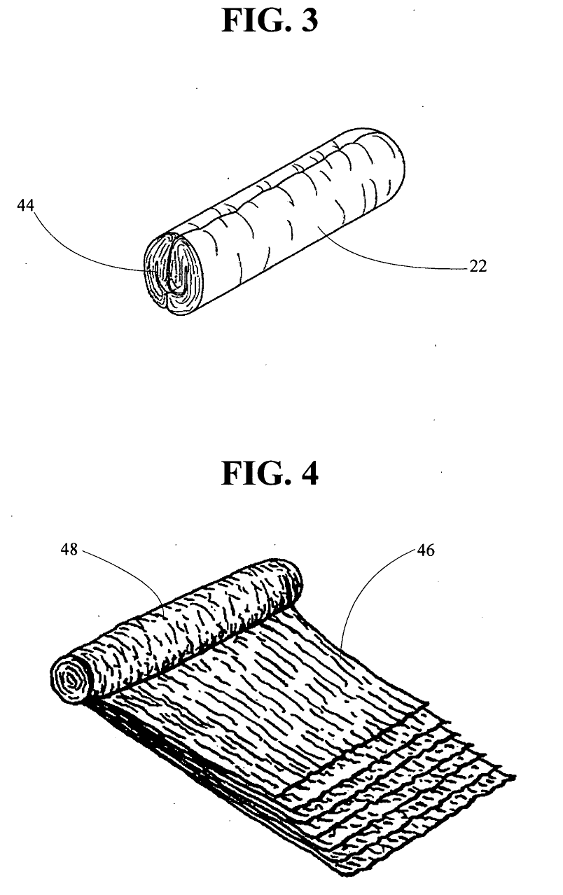

Referring to FIGS. 2 and 3, a method that is suitable for producing the inner core 22 encompasses the following steps: a) the absorbent material is cut to size resulting in the inner core 22 blank 40; and b) the blank 40 is pressed between jaws 42 that can be moved towards each other such that folds 44 are formed in the blank 40. In the preferred embodiment, the compressed inner core blank 40 is of an elongated cylindrical shape having a transverse dimension in the range of between ¼ inch and ½ inch to provide adequate space between the teeth. Pressures and temperatures suitable for compression of the blank 40 are well known in the art. This is but one embodiment of one method of forming the inner core 22. A variety of other techniques are known and acceptable for compressing the blank 40.

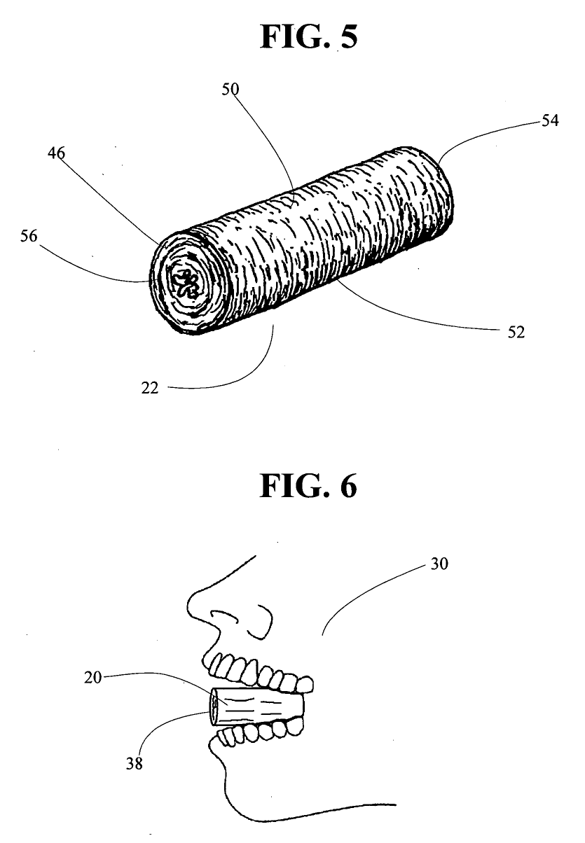

In an alternative embodiment, cotton gauze 46 is rolled into an elongate roll 48. Referring to FIGS. 4 and 5, the elongate roll has a top side 50, a bottom side 52, a first end 54 and an opposing second end 56. The elongate roll 48 can be produced by machine or hand rolling sheets or squares of cotton gauze 46. The tightness of the elongate roll 48 will determine the firmness and compressibility of the cotton gauze 46. It is preferred that under the anticipated force of biting by the patient 30, that when compressed, the elongate roll 48 will have a transverse dimension of at least about ¼ and preferably between the teeth in the range of between about ¼ inch and about ½ inch to provide adequate space between the teeth. The elongate roll 48 will vary in length depending on the patient's 30 teeth and/or gum size.

B. Outer Cover

An outer cover 24 is made a part of the preferred embodiment of the present invention to assure that the fibers of the inner core 22 will not be left behind in the patient's mouth and to maintain the bite block 20 in its intended monolithic structure.

According to the present invention the outer cover is absorbent or semi-absorbent, whereby absorption of saliva or other liquid from within the mouth is reduced compared to bite blocks wherein the absorbent material is in direct contact with the surface. The bite block 20 according to the present invention therefore improves the comfort of using a bite block. The use of a semi-absorbent outer cover 24 is meant that the outer cover 24 has less absorbent capability compared to the absorbent capability of the inner core 22 of the bite block 20. Perforations of the outer cover 24 may allow for absorption of liquid but the absorption by the outer cover 24 will be less than the absorption performed by the inner core 22.

The outer cover 24 is preferably made from a smooth material to avoid friction against the teeth and mouth surface during insertion of the bite block 20, retention of the bite block 20 and removal of the bite block 20. The outer cover 24 may be made from at least one layer of thermoplastics for example polymers such as polyethylene, for example polypropylene, such as polystyrene, for example polyesters, such as polycarbonates, for example nylon, or such as poly(methyl metaacrylate). Likewise, the outer cover may be made in any combinations of mesh, tape, permeable elastomers and thermoplastics.

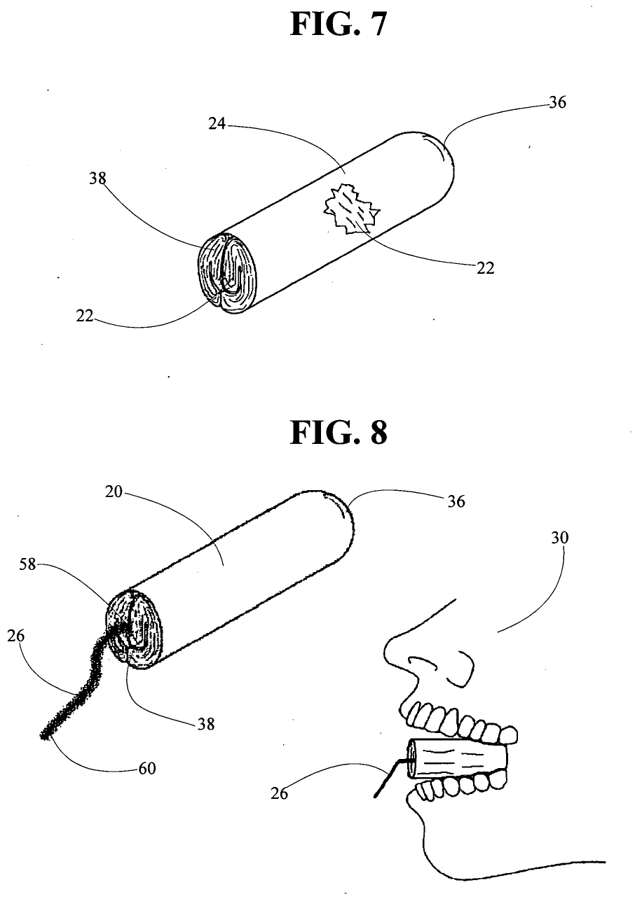

Referring to FIG. 7, the outer cover 24, in the preferred embodiment of the present invention, covers the sides of the bite block 20 and the first end 36 and second end 38 entirely, allowing saliva and other liquid from the patient's mouth to penetrate the out cover 24 and be absorbed by the inner core 22. The permeable outer cover 24 is fastened to at least one layer of the inner core 22. The outer cover 24 may be fastened by a fastening agent, for example by means of contact adhesive or by welding using ultrasound, or heat-assisted melting of the cover material. In particular the fastening may be performed by heat sealing. Another embodiment comprises the use of welded seams, either over the entire area of the outer cover 24 or by fastening in a point-wise fashion. The fastening of the outer cover 24 to the inner core 22 may be performed using a combination of the mentioned means for fastening the outer cover 24.

Fastening of the outer cover 24 to the inner core 22 is in one embodiment performed prior to the “compression” of the bite block 20 into its final shape. In another embodiment, the outer cover 24 is fastened to the inner core 22 after compression of the inner core 22.

C. Finger Tab

Referring to FIG. 8, in one embodiment of the present invention, the bite block 20 contains a flexible withdrawal means 26 to assist in handling of the bite block 20. The withdrawal means 26 will be joined to the inner core 22 of the bite block 10 and will be graspable for removal from the patient's 30 mouth after use. The withdrawal means 26 has a first end 58 and an opposing second end 60. The first end 58 of the withdrawal means 26 is joined to the inner core 22 of the bite block 20 with the opposing second end 60 extending beyond the bite block 20. The withdrawal means 26 can be in the form of string, ribbon, loop, tab or the like. The withdrawal means 26 is attached to the inner core 22 of the bite block 20 in any suitable manner known in the art including sewing, adhesive, or a combination of known bonding methods.

In use, as illustrated in FIG. 8, the first end 54 of the bite block 20 is inserted into the patient's 30 mouth so that it rests between the upper and lower molars. The second end 56 of the bite block 20 extends outside of the patient's 30 mouth. When the patient 30 attempts to close his or her teeth, the patient's 30 teeth will come into contact with the outer cover 24, and the underling inner core 22, of the bite block 20. The space maintained between the upper and lower teeth through use of the soft bite block 20 provide sufficient support to hold the teeth spaced adequately apart for positioning a tube between the front teeth and prevent collapse or compression of the LMA or ETT and hold the mouth open to allow suctioning of oral secretions.

D. Package of the Present Invention

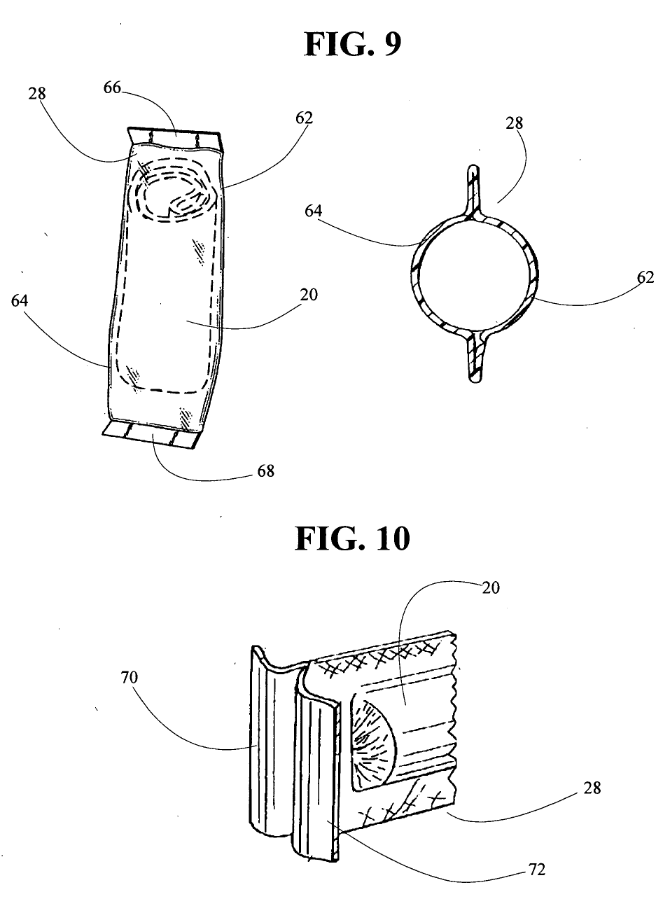

As shown in FIGS. 9 and 10, in the preferred embodiment, each soft bite block 20 is contained within an aseptic or sterile package or pouch 28. The bite block package 28 has a length longer than said axial length 32 of the bite block 20 and a width as greater than the circumference 34 of the bite block 20. The packaging 28 preferably includes an extensible, thermoformed, longitudinal bottom web 62 designed to snugly hug the soft bite block 20, a top web 64 which forms the opposing longitudinal side of the package 28, and opposing ends 66, 68. A peelable heat seal, preferably a hermetic seal, may be provided. Use of hermetic longitudinal seals and hermetic cross-seals will insure that the soft bite block 20 is entirely, hermetically enclosed within the package 28.

“Peelable” refers to a seal which may be readily, manually broken by the consumer. Referring to FIG. 10, unsealed opposing flaps 70A, 70B may be provided at one end of the package 28 to facilitate peeling open the heat sealed package 28.

The packaging 28 can be of any desired thickness, commensurate with the intended use. Generally, the packaging is flexible and fluid-impermeable. Typically, the walls of the package 28 have a thickness of from about. 0127 mm (0.5 mil) to about 0.127 mm (5.0 mils). The packaging 28 can be made of laminates of plastic and paper, or paper fibers with fillers.

Although the best mode contemplated by the inventors for carrying out the present invention is disclosed above, practice of the present invention is not limited thereto. It will be manifest that various additions, modifications and rearrangements of the features of the present invention may be made without deviating from the spirit and scope of the underlying inventive concept.

The individual components mentioned herein need not be fabricated from the disclosed materials, but could be fabricated from virtually any suitable materials.

Moreover, the individual components need not be formed in the disclosed shapes, or assembled in the disclosed configuration, but could be provided in virtually any shape, and assembled in virtually any suitable configuration.

Index of Elements for Soft Bite Block

| | |
|---|---|
| 20 | Soft Bite Block |
| 22 | Inner Core |
| 24 | Outer Cover |
| 26 | Withdrawal Means |
| 28 | Packaging |
| 30 | Patient |
| 32 | Bite Block Axial Length |
| 34 | Bite Block Circumference |
| 36 | Bite Block First End |
| 38 | Bite Block Second End |
| 40 | Inner Core Blank |
| 42 | Compression Jaws |
| 44 | Blank Folds |
| 46 | Cotton Gauze |
| 48 | Elongate Roll |
| 50 | Top Side |
| 52 | Bottom Side |
| 54 | First End |
| 56 | Second End |
| 58 | Withdrawal Means First End |
| 60 | Withdrawal Means Second End |
| 62 | Package Bottom Web |
| 64 | Package Top Web |
| 66 | Package Opposing End |
| 68 | Package Opposing End |
| 70 | Package Unsealed Opposing Flap |
| 72 | Package Unsealed Opposing Flap |

What is claimed is:

1. A soft bite block comprising:
an inner core comprised of a substantially cylindrical mass of compressed absorbent fibers;
an outer cover which covers the outside perimeter of the inner core and maintains the inner core in its substantially cylindrical shape;
a withdrawing means attached to and extending from the inner core to assist in the removal of the soft bite block from a patient's mouth; and
an aseptic or sterile package or pouch for maintaining the soft bite block in an uncontaminated condition prior to use.

2. The bite block of claim 1 wherein the absorbent fibers may be selected from a variety of natural liquid-absorbing materials commonly known in the art and used in absorbent articles such as cellulosic materials for example cotton, rayon (viscose), wood pulp, crepped cellulose wadding, and cross-linked cellulosic fibers.

3. The bite block of claim 1 wherein the outer cover is comprised of an absorbent or semi-absorbent material including any combinations of mesh, tape, permeable elastomers and thermoplastics.

4. The bite block of claim 1 wherein the outer cover is fastened to the exterior of the inner core by a fastening agent, for example by means of contact adhesive or by welding using ultrasound, or heat-assisted melting of the outer cover.

5. The bite block of claim 1 wherein the withdrawal means can take on the form of a ribbon, tab, loop or can consist of an extra-oral section of the soft bite block.

6. The bite block of claim 1 wherein the inner core and cover may be any suitable size and thickness which is suitable for insertion in the patient's mouth and adequate to hold the teeth spaced apart sufficient under biting load.

7. A method of forming the absorbent inner core of a soft bite block, said method comprising the steps of:
a. absorbent material selected from a variety of natural liquid-absorbing materials commonly known in the art and used in absorbent articles such as cellulosic materials for example cotton, rayon (viscose), wood pulp, crepped cellulose wadding, cross-linked cellulosic fibers formed into a fabric, web, or batt is cut to a suitable shape and size to form an inner core blank; and
b. the inner core blank is pressed between jaws that can be moved towards each other with the blank being compressed into an elongated cylindrical shape having a transverse dimension in the range of between ¼ inch and ½ inch to provide adequate space between the teeth.

8. A method as defined in claim 7 wherein pressures and temperatures suitable for compression of the inner core blank are well known in the art.

9. A method of forming a soft bite block, said method comprising the steps of:
a. absorbent material selected from a variety of natural liquid-absorbing materials commonly known in the art and used in absorbent articles such as cellulosic materials for example cotton, rayon (viscose), wood pulp, crepped cellulose wadding, cross-linked cellulosic fibers formed into a fabric, web, or batt is cut to a suitable shape and size to form an inner core blank; and
b. the inner core blank is pressed between jaws that can be moved towards each other with the blank being compressed into an elongated cylindrical shape having a transverse dimension in the range of between ¼ inch and ½ inch to provide adequate space between the teeth; and
c. an outer cover comprised of an absorbent or semi-absorbent material including any combinations of mesh, tape, permeable elastomers and thermoplastics is fastened to the exterior surface of the inner core by a fastening agent, for example by means of contact adhesive or by welding using ultrasound, or heat-assisted melting of the outer cover; and
d. the soft bite block comprised of the compressed inner core and outer cover is placed within a sealed package to maintain the soft bite block in an uncontaminated condition prior to use.

10. A soft bite block comprising:
an inner core comprised of cotton gauze rolled into a substantially cylindrical shape;
an outer cover which covers the outside perimeter of the inner core and maintains the inner core in its substantially cylindrical shape;
a withdrawing means attached to and extending from the inner core to assist in the removal of the soft bite block from a patient's mouth; and
an aseptic or sterile package or pouch for maintaining the soft bite block in an uncontaminated condition prior to use.

11. The bite block of claim 10 wherein the outer cover is comprised of an absorbent or semi-absorbent material including any combinations of mesh, tape, permeable elastomers and thermoplastics.

12. The bite block of claim 10 wherein the outer cover is fastened to the exterior of the inner core by a fastening agent, for example by means of contact adhesive or by welding using ultrasound, or heat-assisted melting of the outer cover.

13. The bite block of claim 10 wherein the withdrawal means can take on the form of a ribbon, tab, loop or can consist of an extra-oral section of the soft bite block.

14. The bite block of claim 10 wherein the inner core and cover may be any suitable size and thickness which is suitable for insertion in the patient's mouth and adequate to hold the teeth spaced apart sufficient under biting load.
